(19) United States
(12) Patent Application Publication
(10) Pub. No.: US 2006/0206175 A1
(43) Pub. Date: Sep. 14, 2006

(54) VESTIBULAR REHABILITATION UNIT

(75) Inventors: Nicolas Fernandez Tournier, Montevideo (UY); Hamlet Suarez, Montevideo (UY); Alejo Suarez, Montevideo (UY)

Correspondence Address: SUGHRUE MION, PLLC, 2100 PENNSYLVANIA AVENUE, N.W., SUITE 800, WASHINGTON, DC, 20037, US

(73) Assignee: TRENO CORPORATION

(21) Appl. No.: 11/383,059

(22) Filed: May 12, 2006

Related U.S. Application Data
(63) Continuation of application No. PCT/IB04/03797, filed on Nov. 15, 2004.

(30) Foreign Application Priority Data
Nov. 14, 2003 (UY) 28083

(51) Int. Cl. A61N 5/06; A61M 21/00

(52) U.S. Cl. 607/88; 600/27

(57) ABSTRACT

An apparatus and method for enabling selective stimulation of oculomotor reflexes involved in retinal image stability. The apparatus enables real-time modification of auditory and visual stimuli according to the patient's head movements, and allows the generation of stimuli that integrate vestibular and visual reflexes. The use of accessories allow the modification of somatosensory stimuli to increase the selective capacity of the apparatus. The method involves generation of visual and auditory stimuli, measurement of patient response and modification of stimuli based on patient response.

The present application is a continuation of PCT application No. PCT/IB2004/003797 filed Nov. 15, 2004, and claims priority from Uruguayan Application No. 28083 filed on Nov. 14, 2003, which applications are incorporated herein by reference, the contents of which are incorporated herein by reference.

### BACKGROUND OF THE INVENTION

1. Field of the Invention

The present invention generally relates to the application of computer technology (hardware and software) to the field of medicine. More specifically, the present invention relates to a Vestibular Rehabilitation Unit for treatment of balance disorders of distinct origin.

2. Description of the Related Art

A patient diagnosed with an episode of vestibular neuronitis experiences symptoms characterized by a prolonged crisis of vertigo, accompanied with nausea and vomiting. Once the acute episode remits, a sensation of instability of a non-specific nature persists in the patient, especially when moving or in spaces where there are many people. The sensation of instability affects the quality of life and increases the risk of falling, especially in the elderly, with all the ensuing complications, including the loss of life.

The mechanism underlying this disorder is a deficit in the vestibulo-oculomotor reflex, aftereffects of the deafferentiation of one of the balance receptors, the vestibular receptor, situated in the inner ear. The procedure to treat this deficit involves achieving a compensation of the vestibular system by training the balance apparatus through vestibular rehabilitation. In order to achieve this compensation, stimulation of the different systems that control the movement of the eyes is performed, as well as stimulation of the somatosensory receptors, the remaining vestibular receptor and the interaction between these components.

Other rehabilitation systems applying virtual reality, for example BNAVE (Medical Virtual Reality Center—University of Pittsburgh) and Balance Quest (Micromedical Technologies), are unable to perform real-time modification of stimuli according to the patient's head movements.

### SUMMARY OF THE INVENTION

The Vestibular Rehabilitation Unit (VRU) enables selective stimulation of oculomotor reflexes involved in retinal image stability. The VRU allows generation of stimuli through perceptual keys, including the fusion of visual, vestibular and somatosensory functions specifically adapted to the deficit of the patient with balance disorders. Rehabilitation is achieved after training sessions where the patient receives stimuli specifically adapted to his/her condition.

Using computer hardware and software, the Vestibular Rehabilitation Unit (VRU) enables real-time modification of stimuli according to the patient's head movements. This allows the generation of stimuli that integrate vestibular and visual reflexes. Moreover, the use of accessories that allow the modification of somatosensory stimuli increases the system's selective capacity. The universe of stimuli that can be generated by the VRU results from the composition of ocular and vestibular reflexes and somatosensory information. This enables the attending physician to accurately determine which conditions favor the occurrence of balance disorders or make them worse, and design a set of exercises aimed at the specific rehabilitation of altered capacities.

The aim of the Vestibular Rehabilitation Unit is to achieve efficient interaction among the senses by controlled generation of visual stimuli presented through virtual reality lenses, auditory stimuli that regulate the stimulation of the vestibular receptor through movements of the head captured by an accelerometer and interaction with the somatosensory stimulation through accessories, for example, but not limited to, an elastic chair and Swiss balls.

The software includes basic training programs. For each program, the Vestibular Rehabilitation Unit can select different characteristics to be associated with a person and a particular session, with the capacity to return whenever necessary to those characteristics that are set by defect.

The Vestibular Rehabilitation Unit also has a web mode that enables it to work remotely from the patient.

### BRIEF DESCRIPTION OF THE DRAWINGS

Reference should be made to the following detailed description which should be read in conjunction with the following figures, wherein like numerals represent like parts:

FIG. 1 is a block diagram illustrating an exemplary embodiment of a Vestibular Rehabilitation Unit; and

FIG. 2 is a flow chart illustrating the training process.

### DETAILED DESCRIPTION

The Vestibular Rehabilitation Unit (VRU) combines a computer, at least one software application operational on the computer, a stimulus generating system, a virtual reality visual helmet and a multidirectional elastic chair, for example, but not limited to, a set of Swiss balls. The system counts with a module for the calibration of the virtual reality visual helmet to be used by the patient.

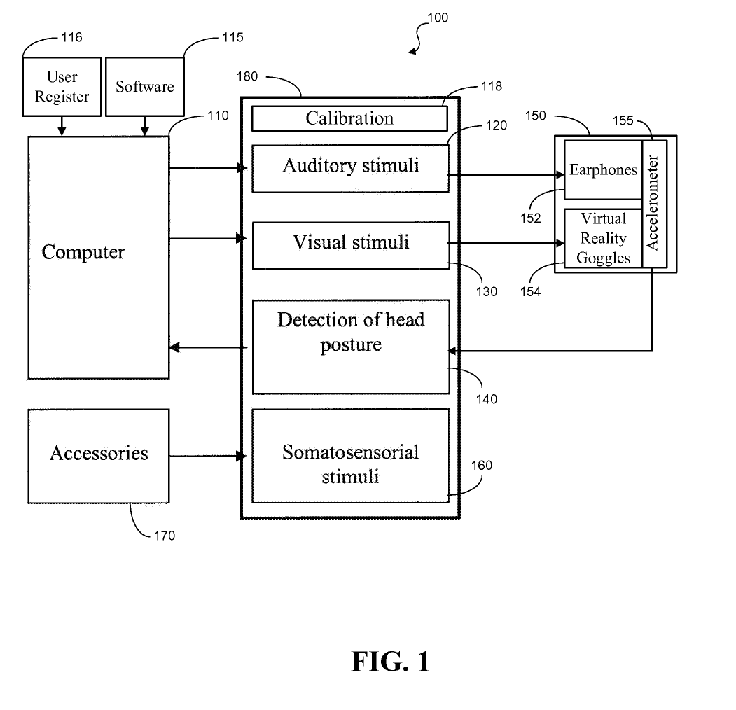

FIG. 1 is a block diagram illustrating an exemplary embodiment of a Vestibular Rehabilitation Unit.

The VRU 100 includes a computer 110, at least one software application 115 operational on the computer, a stimulus generating system 180 including a calibration module 118, an auditory stimuli module 120, a visual stimuli module 130, a head posture detection module 140, and a somatosensorial stimuli module 160, a virtual reality helmet 150, and related system accessories 170, for example, but not limited to, a mat, an elastic chair and an exercise ball. The virtual reality helmet 150 may further include virtual reality goggles 152 and earphones 154.

The software 115 may be embodied on a computer-readable medium, for example, but not limited to, magnetic storage disks, optical disks, and semiconductor memory, or the software 115 may be programmed in the computer 110 using nonvolatile memory, for example, but not limited to, nonvolatile RAM, EPROM and EEPROM.

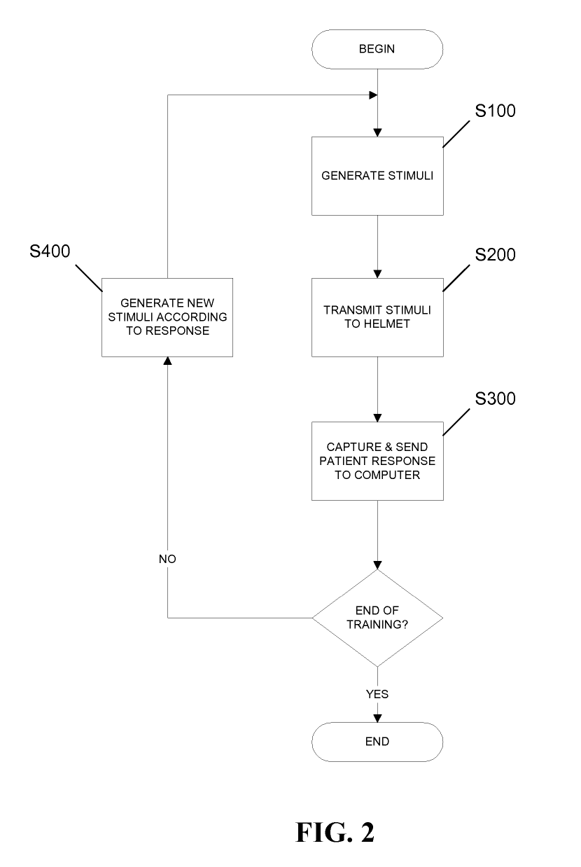

FIG. 2 is a flow chart illustrating the training process. The training process involves generating stimuli S100 by the software 115 and delivering the stimuli to the patient S200 through the virtual reality helmet 150. The response of the patient to this stimuli is captured and sent S300 by the virtual reality helmet 150 to the computer 110 where the software 115 generates new stimuli according to the detected response S400.

The software 115 generates stimuli to compensate for deficiencies detected in the balance centers of the inner ear through sounds and moving images generated in the virtual reality visual helmet 150 and interacts with the sounds and moving images to obtain more efficient stimuli. The software includes at least the following six basic training programs: sinusoidal foveal stimulus, in order to train the slow ocular tracking; random foveal stimulus in order to train the saccadic system; retinal stimulus in order to train the optokinetic reflex; visual-acoustic stimulus in order to treat the vestibular-oculomotor reflex; visual-acoustic stimulus in order to treat the visual suppression of the vestibular-oculomotor reflex; and visual-acoustic stimulus in order to the treat the vestibular-optokinetic reflex.

For each program, the VRU 100 can select different characteristics to be associated with a person and a particular session, with the capacity to return whenever necessary to those characteristics that are set by defect. The characteristics to be determined according to a program may include: duration (in seconds); form of a figure (sphere or circle); size; color (white, blue, red or green that will be seen on a black background); direction (horizontal, vertical); mode (position on the screen, position of the edges, sense); amplitude (in degrees); and frequency (in Hertz).

Auditory and visual stimuli are delivered from the auditory stimuli module 120 and the visual stimuli module 130, respectively, to the patient wearing the virtual reality helmet 150 through the virtual reality goggles 152. The computer 100 generates visual stimuli on the displays of the virtual reality goggles 152 and auditory stimuli in the earphones 154. The implementation of auditory and visual stimuli through a virtual reality helmet 150 enables the isolation of the patient from other environmental stimuli thus achieving high specificity.

Exercises are specified for the patient during some of which the patient is asked to move the head either horizontally or vertically. The detection of the head posture is made by an accelerometer 155 (head tracker) attached to the helmet 150. The accelerometer 155 detects the head's horizontal and vertical rotation angles with respect to the resting position with the eyes looking forward horizontally.

The somatosensory stimuli are generated by the patient him/herself during exercise. The exercises may be performed using the accessories 170. These stimuli may be: stationary gait movements on a firm surface or a soft surface, for example, but not limited to, a mat; and vertical movements sitting on a ball designed for therapeutic exercise, for example, but not limited to, an elastic chair and a set of Swiss balls.

The work with the elastic chair or the Swiss balls selectively stimulates one of the parts of the inner ear involved in balance, whose function is to sense the lineal accelerations, in general gravity. In this way, when the person seated on a ball “bounces” or “rebounds,” they are stimulating the macular, utricule and/or saccule receptors and at the same time interacting with the visual stimuli generated by the software and shown through the virtual reality lenses. The movements that should be performed are specified in accordance with the visual stimulus presented, thereby training the different vestibulo-oculomotor reflexes which are of significant importance for the correct function of the system of balance.

The VRU 100 is capable of generating different stimuli for selective training of the oculomotor reflexes involved in balance function. For algorithm description purposes it is assumed that displays of the virtual reality goggles 152 cover the patient's entire visual field. Stimuli are the result of displaying easily recognizable objects. A real visual field is abstracted as a rectangle visualized by the patient in the resting position. Rx and Ry are coordinates of the center of an object in the real field.

When the patient moves his or her head, the accelerometer 155 transmits the posture-defining angles to the computer 110. An algorithm turns these angles into posture coordinates Cx and Cy on the visual field. The object is shown on the displays at Ox and Oy coordinates. The displays of the virtual reality goggles 152 accompany the patient's movements, therefore, according to the movement composition equations 1 and 2:
Rx=Cx+Ox (Equation 1)
Ry=Cy+Oy (Equation 2)
This nomenclature will be used to describe algorithms.

During the exercises involving vestibular information, the patient may be asked to move the head gently. Periodic auditory stimuli of programmable frequency are used to mark the rhythm of the movement. For example, a short tone is issued every second, asking the patient to move the head horizontally so as to match movement ends with sounds. In this case, an approximation to Cx would be Cx=k cos II t.

Three channels are identified: the auditory channel is an output channel that paces the rhythm of the patient's movement; the image channel “O” is an output channel that corresponds to the coordinates of the object on the display; and the patient channel is an input channel that corresponds to the coordinates of the patient's head in the virtual rectangle.

The following sections involve stimuli of horizontal movements of the patient's eye. Stimuli of vertical movements of the patient's eye are similar. In the algorithms it would be enough to replace coordinate ‘x’ by the relevant ‘y’ coordinate.

In all cases a symbol, for example, a number or a letter, that changes at random is shown inside the object. The patient is asked to say aloud the name of the new symbol every time the symbol changes. This additional cognitive exercise, symbol recognition, enables the technician to check whether the patient performs the oculomotor movement. This is useful for voluntary response stimuli such as smooth pursuit eye movement, saccadic system stimulation, vestibulo-oculomotor reflex and suppression of the vestibulo-oculomotor reflex. Duration, shape, color, direction (right-left, left-right, up-down or down-up), amplitude and frequency may be programmed according to the patient's needs.

Following are stimuli that are associated to the different oculomotor reflexes.

TABLE 1
Smooth pursuit eye movement

| | |
|---|---|
| Auditory channel | No signal |
| Patient's channel | No signal (no head movement) |
| Image channel | Ox = k cos 2 Π F t, with a programmable frequency |
| | “F”. |

The stimulus indicated in Table 1 generates a response from one of the conjugate oculomotor systems called “smooth pursuit eye movement command.” The cerebral cortex has a representation of this reflex at the level of the parietal and occipital lobes. Co-ordination of horizontal plane movements occurs at the protuberance (gaze pontine substance), and co-ordination of vertical plane movements occurs at the brain stem in the pretectal area. It has very important cerebellar afferents, and afferents from the supratentorial systems. From a functional standpoint, it acts as a velocity servosystem that allows placing on the fovea an object moving at speeds of up to 30 degrees per second. Despite the movement, the object's characteristics can be defined, as the stimulus-response latency is minimal.

This type of reflex usually shows performance deficit after the occurrence of lesions of the central nervous system caused by acute and chronic diseases, and especially as a consequence of impairment secondary to aging. The generation of this type of stimulation cancels input of information from the vestibulo-oculomotor reflex. Consequently, when there are lesions that alter the smooth pursuit of objects in the space function, training of this system stimulates improvement of its functional performance and/or stimulates the compensatory mechanisms that will favor retinal image stabilization.

TABLE 2
Saccadic system

| | |
|---|---|
| Auditory channel | No signal |
| Patient's channel | No signal (no head movement) |
| Image channel | Ox = k random(n) |
| | Oy = l random(n) |
| | Where random is a generator of random numbers |
| | triggered at every programmable time interval “t”. |

This random foveal stimulus presented in Table 2 stimulates the saccadic system. The object changes its position every ‘t’ seconds (programmable ‘t’). The saccadic system is a position servo system through which objects within the visual field can be voluntarily placed on the fovea. It is used to define faces, reading, etc. Its stimulus-response latency ranges from about 150 to 200 milliseconds.

The cerebral cortex has a representation of this system at the level of the frontal and occipital lobes. The co-ordination of horizontal saccadic movements is similar to that of the smooth pursuit eye movement at the protuberance (gaze pontine substance), and co-ordination for vertical plane movements at the brain stem in the pretectal area. It has cerebellar afferents responsible of pulse-tone co-ordination at the level of the oculomotor neurons. The training of this conjugate oculomotor command improves retinal image stability through pulse-tone repetitive stimulation on the neural networks involved.

TABLE 3
Optokinetic reflex

| | |
|---|---|
| Auditory channel | No signal |
| Patient's channel | No signal (no head movement) |
| Image channel | An infinite sequence of objects is generated that move |
| | through the display at a speed that can be programmed |
| | by the operator. |

The retinal stimulus indicated in Table 3 trains the Optokinetic reflex. It is called retinal stimulus because it is generated on the whole retina, thus triggering an involuntary reflex. The Optokinetic reflex is one of the most relevant to retinal image stabilization strategies and one of the most archaic from the phylogenic viewpoint. This reflex has many representations in the cerebral cortex and a motor co-ordination area in the brain stem.

To trigger this reflex the system generates a succession of images moving in the direction previously set by the technician in the stimulus generating system 180. The perceptual keys (visual flow direction and velocity, and object size and color) are changed to evaluate the behavioral response of the patient to stimuli. These stimuli are generated on the display of the virtual reality goggles 152 and the patient may receive this visual stimulation while in a standing position and also while walking in place.

As this Optokinetic stimulus is permanently experienced by a subject during his/her daily activities, for example, while looking at the traffic on the street, or looking outside while traveling in a car, it can be generated by changing the perceptual keys that trigger the Optokinetic reflex. These perceptual keys are received by the patient in a static situation i.e., in a standing position, and in a dynamic situation, i.e., while walking in place. This reproduces real life situations, where this kind of visual stimulation is received.

The rotation angle of the patient walking in place in the direction of the visual flow, which is normal, or in the opposite or a random direction, will progressively mark various characteristics of postural response and of normal or pathologic gait to this kind of visual stimulation.

TABLE 4
Vestibulo-oculomotor reflex

| | |
|---|---|
| Auditory channel | Programmable frequency tone “F”. |
| Patient's channel | The patient moves the head horizontally matching end |
| | positions with the tone. When the patient is capable of |
| | making a soft movement this may be represented as: |
| | Cx = k cos Π F t, where F is the tone frequency in the |
| | auditory channel. |
| Image channel | Ox = −Cx |

This stimulus of Table 4 trains the vestibulo-oculomotor reflex. The patient moves the head fixing the image of a stationary object on the fovea. The coordinates of the real object do not change, as the algorithm computes the patient's movement detected by the accelerometer, and shows the image after compensating the movement of the head in full.

This allows stimulation of the angular velocity accelerometers located in the crests of the inner-ear semicircular canals. Movement of the patient along the x or y plane, or along a combination of both at random, will generate oculomotor responses that will make the eyes move opposite in phase to the head in order that the subject may be capable of stabilizing the image on the retina when the head moves. According to the algorithm, the VRU system 100 senses, through an accelerometer 155 attached to the virtual reality helmet 150, the characteristics of the patient's head movements (axis, direction and velocity) and generates a stimulus that moves with similar characteristics but opposite in phase. For this reason, the patient perceives the static stimulus at the center of his/her visual field

The VRU program generates symbols (letters and/or numbers) on this stimuli that change periodically and that the patient must recognize and name aloud. This accomplishes two purposes.

First, that the technician controlling the development of the rehabilitation session may verify that the patient is generating the vestibulo-oculomotor reflex that enables him/her to recognize the symbol inside the object. This is especially determining in elderly patients with impaired concentration.

Second, to test the patient's evolution. In numerous circumstances the patient has a deficit of the vestibulo-oculomotor reflex and finds it difficult to recognize the symbols inside the object. In the course of the sessions devoted to vestibulo-ocular reflex training, icon recognition performance begins to improve.

When the subject achieves the compensation of the vestibulo-oculomotor reflex, the percentage of icon recognition is normal. Visual and vestibular sensory information are “fused” in this stimulus to train a reflex relevant to retinal image stabilization

TABLE 5
Suppression of the vestibulo-oculomotor reflex

| | |
|---|---|
| Auditory channel | Programmable frequency tone “F”. |
| Patient's channel | The patient moves the head horizontally matching end |
| | positions with the tone. When the patient is capable of |
| | making a soft movement this may be represented as: |
| | Cx = k cos Π F t, where F is the tone frequency in the |
| | auditory channel. |
| Image channel | Ox = 0 |

Table 5 indicates the stimulus that trains the suppression of the vestibulo-oculomotor reflex. The patient moves the head fixing on the fovea the image of an object accompanying the head movement. This stimulation reproduces the perceptual situation where the visual object moves in the same direction and at the same speed as the head. For this reason, if the vestibulo-ocular reflex is performed, the subject loses reference to the object.

In this situation the vestibulo-oculomotor reflex is “cancelled” by the stimulation of neural networks inhibiting the cerebellum (Purkinje strand) and inhibits the ocular movements opposite in phase to the head movements placing the eye ball “to accompany” head movements. This inhibition is altered in some cerebellar diseases, and the successive exposure to this perceptual situation stimulates post-lesion compensation and adaptation

TABLE 6
Vestibulo-optokinetic reflex

| | |
|---|---|
| Auditory channel | Programmable frequency tone “F”. |
| Patient's channel | The patient moves the head horizontally matching end |
| | positions with the tone. When the patient is capable of |
| | making a soft movement this may be represented as: |
| | Cx = k cos Π F t, where F is the tone frequency in the |
| | auditory channel. |
| Image channel | An infinite sequence of objects is generated that move |
| | through the “real” visual field at a speed that can be |
| | programmed by the operator. |
| | When the patient moves in the same direction, he/she |
| | tries to “fix” the image on the retina. This reflex is |
| | stimulated by the generation of a movement on the |
| | display as follows: |
| | Velocity (Ox) = programmed velocity-velocity (head) |

This stimulus of Table 6 trains the vestibulo-optokinetic reflex. When the patient “follows” the object, its movement on the display slows down. When it moves in the opposite direction, its movement on the displays becomes faster. This type of stimulation has been designed to generate a simultaneous multisensory stimulation in the patient, the perceptual characteristics of which (velocity, direction, etc., of the stimuli) should be measurable and programmable.

The patient must move the head in the plane where the stimulus is generated, and the visual perceptual characteristic received by the patient is modified according to the algorithm. This reproduces real life phenomena, for example, an individual looking at the traffic on a street (optokinetic stimulation) rotates his/her head (vestibular stimulation), and generates an adaptation of the reflex (visual-vestibular reflexes) in order to establish retinal image stability.

In patients showing damage to the sensory receptors or to the neural networks of integration of sensory information the reflex adaptation of this “addition” of sensory information is performed incorrectly and generates instability. The systematic exposure to this visual and vestibular stimulation through different perceptual keys stimulates post-lesion adaptation mechanisms.

This combined stimulation (vestibular and visual) is also generated in the patients through changes in somatosensory information, alteration of the feet support surface (firm floor, synthetic foam of various consistencies). This is a real life sensory probability where the subject may obtain visual-vestibular information standing on surfaces of variable firmness (concrete, grass, sand). This wide spectrum of combined sensory information aims at developing in the patient (who is supported by a safety harness) postural and gait adaptation phenomena in the light of complex situations where sensory information is multiple, for example, an individual going up an escalator or walking in an open space such as a mall, rotating his/her head and at the same time looking at the traffic flow from a long distance, e.g. 100 m. The software generates this “function fusion” to generate combined and simultaneous stimuli of variable complexity and measurable perceptual keys.

The VRU 100 also has a remote mode that enables it to work remotely from the patient over a network, for example, but not limited to, the World Wide Web, a Local Area Network (LAN) and a Wide Area Network (WAN). In these cases, the VRU 100 includes a register of users 116 that permits it to identify those people that it is treating and in this way only changes data pertinent to them and their corresponding training sessions.

It should be emphasized that the above-described embodiments of the present invention are merely possible examples of implementations, merely set forth for a clear understanding of the principles of the invention. Many variations and modifications may be made to the above-described exemplary embodiments of the invention without departing substantially from the spirit and principles of the invention. All such modifications and variations are intended to be included herein within the scope of this disclosure and the present invention and protected by the following claims.

What is claimed is:

1. A Vestibular Rehabilitation Unit, comprising:
a computer;
at least one software application operational on the computer;
a stimulus generating system capable of generating stimuli;
a virtual reality helmet for providing the stimuli to a patient; and
accessories enabling the patient to perform specified exercises.

2. The Vestibular Rehabilitation Unit of claim 1, wherein the virtual reality helmet comprises earphones and virtual reality goggles.

3. The Vestibular Rehabilitation Unit of claim 2, wherein the virtual reality helmet further comprises an accelerometer capable of detecting head movements of a patient.

4. The Vestibular Rehabilitation Unit of claim 3, wherein the stimulus generating system comprises an auditory stimuli module capable of providing audio stimuli to the virtual reality helmet, and visual stimuli module capable of providing visual stimuli to the virtual reality helmet.

5. The Vestibular Rehabilitation Unit of claim 4, wherein the stimulus generating system further comprises a head posture detection module capable of determining head posture of a patient based on accelerometer information.

6. The Vestibular Rehabilitation Unit of claim 5, wherein the stimulus generating system further comprises a somatosensorial stimuli module capable of receiving somatosensorial stimuli generated by a patient performing using the accessories.

7. The Vestibular Rehabilitation Unit of claim 1, wherein the accessories comprise at least one of a hard surface, a mat, an elastic chair and a set of Swiss balls.

8. The Vestibular Rehabilitation Unit of claim 1, wherein the at least one software application comprises at least one vestibular rehabilitation training program.

9. The Vestibular Rehabilitation Unit of claim 8, wherein the at least one vestibular rehabilitation training program comprises at least one of a sinusoidal foveal stimulus program to train the slow ocular tracking, random foveal stimulus program to train the saccadic system, a retinal stimulus program to train the optokinetic reflex, a visual-acoustic stimulus program to treat the vestibular-oculomotor reflex; visual-acoustic stimulus in order to treat the visual suppression of the vestibular-oculomotor reflex, and a visual-acoustic stimulus program to the treat the vestibular-optokinetic reflex.

10. The Vestibular Rehabilitation Unit of claim 1, further comprising a register of users that permits identification of patients to enable the Vestibular Rehabilitation Unit to only change data and corresponding training sessions related to identified patients, wherein the Vestibular Rehabilitation Unit is enabled to work remotely from a patient over a network.

11. A vestibular rehabilitation training process comprising:
generating auditory and visual stimuli using computer software;
delivering the stimuli to a patient through a virtual reality helmet;
capturing patient responses through the virtual reality helmet to the stimuli;
sending the patient responses to the computer; and
generating new stimuli with the computer software according to the patient response.

12. A computer readable medium having embodied therein a program for making a computer execute a vestibular rehabilitation training process, the program including computer executable instructions for performing operations comprising:
generating auditory and visual stimuli using computer software;
delivering the stimuli to a patient through a virtual reality helmet;
capturing patient responses through the virtual reality helmet to the stimuli;
sending the patient responses to the computer; and
generating new stimuli with the computer software according to the patient response.

13. The computer readable medium of claim 12 wherein the medium comprises at least one of magnetic storage disks, optical disks, and semiconductor memory.

14. A computer having programmed therein a program for making a computer execute a vestibular rehabilitation training process, the program including computer executable instructions for performing operations comprising:
generating auditory and visual stimuli using computer software;
delivering the stimuli to a patient through a virtual reality helmet;
capturing patient responses through the virtual reality helmet to the stimuli;
sending the patient responses to the computer; and
generating new stimuli with the computer software according to the patient response.

15. A Vestibular Rehabilitation Unit, comprising:
means for generating auditory and visual stimuli using computer software;
means for delivering the stimuli to a patient through a virtual reality helmet;
means for capturing patient responses through the virtual reality helmet to the stimuli;
means for sending the patient responses to the computer; and
means for generating new stimuli with the computer software according to the patient response.
